(19) United States
(12) Patent Application Publication
(10) Pub. No.: US 2020/0253858 A1
(43) Pub. Date: Aug. 13, 2020

(54) COSMETIC COMPOSITION COMPRISING CITRON OIL BEAD

(71) Applicant: AMOREPACIFIC CORPORATION, Seoul (KR)

(72) Inventors: Byeongbae JEON, Yongin-si (KR); Yewon KIM, Yongin-si (KR); Jisue PARK, Yongin-si (KR); Gusang KWON, Yongin-si (KR)

(73) Assignee: AMOREPACIFIC CORPORATION, Seoul (KR)

(21) Appl. No.: 16/785,183

(22) Filed: Feb. 7, 2020

(30) Foreign Application Priority Data
Feb. 8, 2019 (KR) 10-2019-0015002
Aug. 6, 2019 (KR) 10-2019-0095360

(51) Int. Cl. A61K 8/92; A61K 8/04; A61K 8/85; A61K 8/34; A61K 8/81; A61Q 13/00

(52) U.S. Cl. CPC A61K 8/922; A61K 8/04; A61K 8/85; A61K 2800/10; A61K 8/8111; A61Q 13/00; A61K 8/342

(57) ABSTRACT

One aspect of the present disclosure relates to a cosmetic composition including a citron oil bead. More specifically, the cosmetic composition includes an appropriate amount of a citron oil bead including a citron oil, a behenyl alcohol, a jojoba ester, and a hydrogenated C₆₋₁₄ polyolefin in an appropriate weight ratio. The cosmetic composition improves the preference for the fragrance and stabilize the mind and body with the fragrance to induce a comfortable sleeping, thereby improving the quality of sleep.

### CROSS-REFERENCE TO RELATED APPLICATION

This application claims the benefit of priority based on Korean Patent Application No. 10-2019-0015002, filed on Feb. 8, 2019, and Korean Patent Application No. 10-2019-0095360, filed on Aug. 6, 2019, the entire contents of each of which are incorporated herein by reference.

### TECHNICAL FIELD

One aspect of the present disclosure relates to a cosmetic composition comprising a citron oil bead.

### BACKGROUND ART

As the 21st century enters, the biggest task of mankind is concentrated on the issue of improving the quality of life. To this end, in recent years, emotional engineering studies have been actively conducted to reveal the correlation between products and emotional and physiological conditions by observing the emotional and physiological responses of humans to products domestically. However, these emotion-related studies have mainly been performed on visual sense and auditory sense, but they are still insignificant with respect to the olfactory sense (fragrance).

Since olfactory sense is a chemical sense unlike other senses such as visual sense and auditory sense, not only the precision of stimulus control is required but also the neurotransmission to the brain, psychological emotional evaluation, and physiological changes (central and autonomic nervous systems) are performed at the same time as the stimulation of the fragrance, thereby making the research difficult.

The fragrance that stimulates the olfactory sense has a great influence on the human sensibility with various kinds of fragrance such as fragrant fragrance and unpleasant fragrance. In addition to studies on the effect of the forest bath on the human body and pheromones (inducing substance) related to sexual behavior of the animal, studies on the physiological effects of odors such as ammonia have been recently carried out.

The impulse of the fragrance is transmitted to the cerebral limbic system through the olfactory cell of the nose, and the olfactory emotional and emotional elements are added, thereby causing emotional and physiological changes such as pleasure or disgust, and awakening or drowsiness depending on the quality of the smell. It can be also explained by this phenomenon that a strong mood is induced when a perfume is taken, defensive reflexes such as sneezing occur when a unpleasant smell is taken, and breathing stops reflexively when the smell of ammonia is taken. As such, the olfactory sense plays a major role in the maintenance of human life and the identification of the object that smells.

Sleep is an important physiological function of brain-developed animals and is an indispensable action for survival. Insufficient sleep leads to sleepiness, irritation, and loss of vitality, resulting in poor quality of life. In some cases, it may cause serious disruption to life maintenance. Sleep is a function to prevent such conditions from occurring, so it is very important to have a deep sleep.

Accordingly, in recent years, various types of products have been actively developed by applying the sleep-related functionality to natural oils.

For example, Japanese Patent Application Laying-Open No. 2011-037721 discloses a skin external preparation for improving sleep, comprising an essential oil obtainable from genus citrus plants.

However, until now, the sleep improvement-related products using the natural oil are those utilizing the functionality that the natural oil simply has a positive effect on the improvement of sleep, and thus there has not been developed a sleep improvement-related product, particularly, a product in the cosmetics field, which provides a more detailed sleep improvement effect by deriving the specific combinations and forms of these natural oils.

In order to solve the above problems, the present inventors have mixed a citron oil among natural oils with a behenyl alcohol, a jojoba ester, and a hydrogenated C₆₋₁₄ polyolefin to prepare a bead form, and have found that when a cosmetic composition comprising an appropriate amount of the bead was used before sleep, the cosmetic composition enhanced the preference for the fragrance to stabilize the mind and body, thereby reducing sleep latency, improving sleep efficiency, and decreasing body movement, and thus improved the overall quality of sleep.

Therefore, it is an object of the present invention to provide a cosmetic composition comprising a citron oil bead.

It is another object of the present invention to provide a composition for inducing a deep sleep, comprising a citron oil bead.

### SUMMARY OF THE INVENTION

In order to achieve the above object, one aspect of the present disclosure provides a cosmetic composition comprising a citron oil bead.

The citron oil bead may comprise a citron oil, a behenyl alcohol, a jojoba ester, and a hydrogenated C₆₋₁₄ polyolefin.

The citron oil bead may comprise 10 to 140 parts by weight of behenyl alcohol, 10 to 140 parts by weight of jojoba ester, and 300 to 1000 parts by weight of hydrogenated C₆₋₁₄ polyolefin, based on 100 parts by weight of the citron oil.

The citron oil bead may be comprised in an amount of 2.0 to 10.0% by weight, based on the total weight of the cosmetic composition.

The particle diameter of the citron oil bead may be 0.05 to 0.8 cm.

The cosmetic composition may be formulated into a skin softener (skin toner), a nutritional toner, an emulsion, a massage cream, a nutritional cream, a pack, a gel, skin adhesive type cosmetics, a lotion, an ointment, a gel, a cream, a patch, or a spray formulation.

One aspect of the present disclosure also provides a method for inducing deep sleep, comprising applying a cosmetic composition comprising a citron oil bead.

The cosmetic composition according to the present invention comprises a citron oil in a bead form to enhance the preference for the fragrance of citron oil, thereby being able to exhibit the effect of stabilizing the mind and body and improving the quality of sleep.

Specifically, the cosmetic composition may reduce sleep latency, improve sleep efficiency, and decrease body movement, thereby improving the quality of sleep.

In addition, the citron oil bead included in the cosmetic composition may comprise a citron oil, a behenyl alcohol, a jojoba ester, and a hydrogenated C₆₋₁₄ polyolefin in an appropriate weight ratio to form a suitable bead form as an ingredient of a cosmetic composition, thereby preparing a cosmetic product having excellent quality.

### BRIEF DESCRIPTION OF DRAWINGS

FIG. 1 is a graph showing the evaluated results of the preference for the fragrance for the sleeping packs prepared in the example and comparative example 4.

FIG. 2 is a graph showing the evaluated results of the residual fragrance strength for the sleeping packs prepared in the example and comparative example 4.

FIG. 3 is a graph showing the evaluated results of the image for the overall fragrance for the sleeping packs prepared in the example and comparative example 4.

FIG. 4 is a graph showing the evaluated results of sleep latency for the sleeping packs prepared in the example and comparative examples 5 and 6.

FIG. 5 is a graph showing the evaluated results of sleep efficiency for the sleeping packs prepared in the example and comparative examples 5 and 6.

FIG. 6 is a graph showing the measured results of the number of body movements during sleeping when the sleeping packs prepared in the example and comparative examples 5 and 6 are used.

### DETAILED DESCRIPTION OF THE INVENTION

Hereinafter, the present invention will be described in detail for those skilled in the art to easily practice. However, the present invention may be embodied in many different forms and is not limited to the examples set forth herein.

### Cosmetic Composition

The present invention relates to a cosmetic composition comprising a citron oil bead.

In the present invention, the citron oil bead refers to a bead comprising a citron oil, a behenyl alcohol, a jojoba ester, and a hydrogenated C₆₋₁₄ polyolefin.

The citron oil bead may comprise 10 to 140 parts by weight of behenyl alcohol, 10 to 140 parts by weight of jojoba ester, and 300 to 1000 parts by weight of hydrogenated C₆-₁₄ polyolefin , based on 100 parts by weight of the citron oil. The weight ratio is optimized so that a bead form is well formed. In addition, by forming the citron oil in the form encapsulated by a bead form, it is optimized in consideration of the extent to which the fragrance derived from citron oil may be expressed so as to induce the stability of the mind and body.

In the present invention, the citron oil is a yellow liquid obtained by pressing the peel of natural citron, which is rich in vitamin C and is effective in relieving the fatigue and stress accumulated on the skin due to the antioxidant effect; which contains a hesperidin ingredient that protects the capillary blood vessels and is effective in preventing cerebrovascular disorders and stroke; and which also has the efficacy of discharging the waste accumulated in the body through drainage and excretion action. Wherein, the natural citron means the citron fruit grown and harvested from citron tree, not the processed type of citron.

The citron oil may have more improved preference for the fragrance due to the form encapsulated by a bead compared with the fragrance of the citron oil itself, and thus, is preferably comprised in the cosmetic composition in the form of citron oil bead.

If the content of the citron oil is lower in the appropriate weight ratio of the four ingredients comprised in the citron oil bead as described above, the fragrance of the citron oil is insignificant and the improvement of the preference for the fragrance is insignificant, and thus the effects of stabilizing the mind and body and improving the quality of sleep may be lowered. If the content of the citron oil is excessive, it may be difficult to form a bead form, and the fragrance of the citron oil is excessively strong or deteriorated, which may lower the preference for the fragrance.

In addition, the content of the citron oil may be 0.1 to 1.4% by weight, preferably 0.2 to 1.2% by weight, more preferably 0.3 to 1.0% by weight, based on the total weight of the cosmetic composition. If the content of the citron oil in the cosmetic composition satisfies the above-mentioned range, the cosmetic composition may enhance the preference for the fragrance to stabilize the mind and body, and thus inducing a deep sleep.

In the present invention, the behenyl alcohol is a naturally derived fatty alcohol ingredient obtained from coconut and palm oil. The behenyl alcohol is a naturally derived fatty alcohol ingredient that is not a chemical alcohol ingredient, and may have a moisturizing function to prevent moisture loss in the skin to maintain moistness and a function to control the viscosity of a cosmetic composition.

In the four ingredients comprised in the citron oil bead as described above, the behenyl alcohol may be comprised in an amount of 10 to 140 parts by weight, preferably 50 to 140 parts by weight, more preferably 80 to 120 parts by weight, based on 100 parts by weight of the citron oil bead. If it is less than the above-mentioned range, the viscosity of the cosmetic composition is lowered, which may make it difficult to form a bead form and may deteriorate a moisturizing function of the cosmetic composition. If it is more than the above-mentioned range, the viscosity of the cosmetic composition is excessive high, which may lower the feeling of use.

In the present invention, the jojoba ester may be a jojoba oil, a hydrogenated jojoba oil, or a complex mixture of esters obtained by subjecting mixture thereof to an ester exchange reaction. The jojoba ester may act as a skin-softening agent to soften the texture of the skin softly and smoothly, and may prevent the evaporation of moisture in the skin to help to maintain a moist skin.

In the four ingredients comprised in the citron oil bead as described above, the jojoba ester may be comprised in an amount of 10 to 140 parts by weight, preferably 50 to 140 parts by weight, more preferably 80 to 120 parts by weight, based on 100 parts by weight of the citron oil bead. If it is less than the above-mentioned range, the moisturizing function of the cosmetic composition may be deteriorated. If it is more than the above-mentioned range, it may be difficult to form a bead form.

In the present invention, the hydrogenated C₆₋₁₄ polyolefin is comprised in the cosmetic composition, thereby being able to prevent the moisture in the skin from evaporating to help in maintaining skin moisturizing ability and being able to function as a non-water-soluble thickener to act to increase the viscosity of a cosmetic composition.

In the four ingredients comprised in the citron oil bead as described above, the hydrogenated C₆₋₁₄ polyolefin may be comprised in an amount of 300 to 1000 parts by weight, preferably 300 to 800 parts by weight, more preferably 400 to 600 parts by weight, based on 100 parts by weight of the citron oil bead. If it is less than the above-mentioned range, the viscosity of the cosmetic composition is lowered, which may make it difficult to form a bead form and may deteriorate a moisturizing function of the cosmetic composition. If it is more than the above-mentioned range, the viscosity of the cosmetic composition is excessive high, thereby being able to lower the feeling of use.

In the present invention, the citron oil bead comprising the citron oil, behenyl alcohol, jojoba ester, and hydrogenated C₆₋₁₄ polyolefin as described above may be comprised in an amount of 2.0 to 10.0% by weight, preferably 2.0 to 8.0% by weight, more preferably 2.0 to 6.0% by weight, based on the total weight of the cosmetic composition. If it is less than the above-mentioned range, the effects of stabilizing the mind and body and improving the quality of sleep due to the improved preference for the fragrance may be insignificant. If it is more than the above-mentioned range, the preference for the fragrance may be lowered.

In the present invention, the particle diameter of the citron oil bead may be 0.05 to 0.8 cm. The particle diameter may preferably be 0.1 to 0.6 cm, more preferably 0.2 to 0.5 cm. If the particle diameter of the citron oil bead is included within the above-mentioned range, the uniformity of the beads is increased, thereby obtaining a cosmetic composition having excellent quality. If it is less than the above-mentioned range, the content of the citron oil comprised in the citron oil bead is small and the improvement of the preference for the fragrance is insignificant, and thus the effects of stabilizing the mind and body and improving the quality of sleep may be insignificant. If it is more than the above-mentioned range, the feeling of use may be poor.

In the present invention, the cosmetic composition is formulated into a skin softener, a nutritional toner, an emulsion a massage cream, a nutritional cream, a pack, a gel, skin adhesive type cosmetics, a lotion, an ointment, a gel, a cream, a patch, or a spray formulation.

The cosmetic composition according to the present invention may be more effective in improving the quality of sleep, especially when it is formulated to be applied to the skin before sleeping. For example, the cosmetic composition may be formulated into a sleeping pack.

In the present invention, the cosmetic composition comprising a citron oil bead may further comprise a ingredient to be normally compounded in a cosmetic composition, for example, a surfactant, a water-soluble polymer, a powder other than the above-mentioned specific red complex powder and each pigment, a moisturizer, a preservative, a medicinal ingredient, an ultraviolet absorber, a pigment, an inorganic salt or an organic acid salt, a perfume, a chelating agent, a pH adjusting agent, water, and the like, within a range not to impair the effect of the present invention, if necessary.

Examples of the surfactant include surfactants commonly used in cosmetics, for example, nonionic surfactants such as polyoxyethylene alkyl ethers, polyoxyethylene fatty acid esters, polyoxyethylene sorbitan fatty acid esters, glycerin fatty acid esters, polyglycerin fatty acid esters, polyoxyethylene glycerin fatty acid esters, polyoxyethylene hydrogenated castor oil, and polyoxyethylene sorbitol fatty acid esters; anionic surfactants represented by fatty acid soaps such as sodium stearate and triethanolamine palmitate; cationic surfactants; amphoteric surfactants, and the like.

Examples of the water-soluble polymer include water-soluble polymers commonly used in cosmetics, such as carboxymethylcellulose, methylcellulose, hydroxymethylcellulose, polyvinyl alcohol, polyvinylpyrrolidone, tragacanth gum, carrageenan, locust bean gum, dextrin, dextrin fatty acid ester, carboxyvinyl polymer, xanthan gum, gelatin, sodium alginate, gum arabic, water-soluble collagen, and the like.

Examples of the moisturizer include a moisturizer commonly used in cosmetics, such as sorbitol, xylitol, glycerin, maltitol, propylene glycol, 1,3-butylene glycol, 1,4-butylene glycol, sodium pyrrolidonecarboxylate, lactic acid, sodium lactate, polyethylene glycol, and the like.

Examples of the preservative include preservatives commonly used in cosmetics, such as paraoxybenzoic acid alkyl ester, sodium benzoate, potassium sorbate, and the like. Examples of the medicinal ingredient include ingredients commonly used in cosmetics, such as vitamins, herbal medicines, anti-inflammatory agents, bactericides, and the like.

Examples of the ultraviolet absorber include ultraviolet absorbers commonly used in cosmetics, such as para-aminobenzoic acid-based ultraviolet absorbers, anthranil-based ultraviolet absorbers, salicylic acid-based ultraviolet absorbers, cinnamic acid-based ultraviolet absorbers, benzophenone-based ultraviolet absorbers, and the like.

Examples of the pigment include pigments applicable to cosmetics, such as natural pigments, for example, alga Haematococcus pigment, and the like.

In addition, salts may be used as an emulsion stabilizer.

The cosmetic composition of the present invention as described above may stabilize the mind and body due to the improved preference for the fragrance, thereby improving the quality of sleep. The quality of sleep may be improved, for example, by reducing sleep latency, improving sleep efficiency, and reducing body movement.

In addition, in the case where the cosmetic composition is commercialized as a product to be used before sleeping, when the preference for the fragrance is increased while the fragrance of natural citron comprised in the citron oil bead goes out and the fragrance is kept for along time, the fragrance of the natural citron stabilizes the mind and body and induces a comfortable sleeping, thereby improving the quality of sleep.

Therefore, the cosmetic composition of the present invention may be more advantageous in improving the quality of sleep when it is formulated into a formulation to be used before sleeping, for example, a sleeping pack.

### Method for Preparing Cosmetic Composition

The present invention also relates to a method for preparing a cosmetic composition comprising a citron oil bead.

A method for preparing the cosmetic composition may comprise the steps of: (S1) mixing a citron oil, a behenyl alcohol, a jojoba ester, and a hydrogenated C₆₋₁₄ polyolefin to form a citron oil bead; and (S2) preparing a cosmetic composition using the citron oil bead.

Specifically, in step (S1), the citron oil bead may be formed by heating the four ingredients at a temperature of 70 to 100° C. for 3 to 10 minutes, mixing them, and then drying the mixture.

The temperature may be 70 to 100° C., preferably 75 to 95° C., more preferably 80 to 90° C. If the temperature is less than 70° C., the raw materials may not be sufficiently mixed. If the temperature is more than 100° C., the physical properties of the raw materials may be denatured and it may be difficult to form a bead form.

The heating and mixing time may be 3 to 10 minutes, preferably 4 to 9 minutes, and more preferably 5 to 8 minutes. If the time is less than 3 minutes, the raw materials may not be sufficiently mixed. If the time is more than 10 minutes, there may be no additional advantage due to overtime.

At this time, the citron oil, behenyl alcohol, jojoba ester, and hydrogenated C₆₋₁₄ polyolefin is used in the same weight ratio as described above.

In step (S2), the cosmetic composition may be prepared by applying to the citron oil beads raw materials and methods, which are commonly used in the art in order to prepare a specific formulation according to the formulation of the cosmetic composition to be manufactured.

For example, an emulsion-type cosmetic composition comprising the citron oil bead may be prepared by emulsification using the raw materials and the citron oil beads, which are used when preparing a general emulsion-type cosmetic composition.

### Composition for Inducing a Deep Sleep

The present invention also relates to a composition for inducing a deep sleep, comprising the citron oil bead. The composition of the citron oil bead is the same as described above.

In the present invention, a case where a deep sleep is not had includes a wide range of cases of suffering mental and physical difficulties associated with sleep, such as a phenomenon that does not take a healthy sleep, for example, having a light sleep or waking up several times during sleep, and a phenomenon that does not maintain awakening during the day despite having enough sleep.

In the present invention, the citron oil bead may be comprised in an amount of 2.0 to 10.0% by weight, preferably 2.0 to 8.0% by weight, more preferably 2.0 to 6.0% by weight, based on the total weight of the composition for inducing a deep sleep. If the content of the citron oil bead is less than 2.0% by weight, the effects of stabilizing the mind and body and improving the quality of sleep due to the improvement of the preference for the fragrance may be insignificant. If the content of the citron oil bead is more than 10.0% by weight, the preference for the fragrance is lowered, and thus the effect of inducing a deep sleep may be lowered.

The preference for the fragrance of the citron oil bead is excellent. In particular, the fragrance and fragrance strength of the citron oil bead relieve stress, provide psychological stability, and allow a person to fall asleep comfortably, and thus being able to induce a deep sleep.

Specifically, due to the fragrance of the citron oil bead, the composition for inducing a deep sleep, comprising the citron oil bead may reduce sleep latency to achieve excellent sleep efficiency, and decrease body movement to improve sleep quality, thereby inducing a deep sleep.

In addition, the composition for inducing a deep sleep may be formulated into a skin softener, a nutritional toner, an emulsion, a massage cream, a nutritional cream, a pack, a gel, skin adhesive type, a lotion, an ointment, a gel, a cream, a patch, or a spray formulation. If a formulation may be applied to the skin, it is not limited.

### Method for Inducing a Deep Sleep

The present invention also relates to a method for inducing a deep sleep. The method for inducing a deep sleep comprises the step of applying the composition for inducing a deep sleep, comprising the citron oil bead as described above, to a subject who does not have a deep sleep due to sleep disorder.

At this time, the composition for inducing a deep sleep may be applied to the skin, preferably the facial skin of a subject who does not have a deep sleep. When the composition is applied to the facial skin, the stability of the mind and body is obtained from the fragrance of the citron oil bead, and thus it may be more advantageous to have a deep sleep.

Further, the composition for inducing a deep sleep may be applied to the skin once a day, preferably before sleeping. Since the fragrance of citron oil beads included in the composition may induce sleeping and take a deep sleep, it is advantageous to apply to the skin before sleeping to induce a deep sleep.

Furthermore, the composition for inducing a deep sleep may exhibit the effect of inducing a deep sleep when applied to the skin once a day for 3 to 8 weeks. The composition for inducing sleep exhibits the effect of inducing a deep sleep even when applied to the skin once a day, and generally may exhibit the effect of inducing a very excellent deep sleep when used continuously for 3 weeks or more, preferably 3 to 8 weeks.

In addition, the amount of one application of the composition for inducing a deep sleep inducing may generally vary from 25 mg to 150 mg, depending on the condition of a subject who does not have a deep sleep, degree of sleep disorder, application area and form of composition. The composition may be suitably regulated to be applied within the range of the above-mentioned one application amount.

Hereinafter, preferred examples will be provided to help understanding the present invention, but the following examples are only for illustrating the present invention. It will be apparent to those skilled in the art that various changes and modifications may be made within the scope and spirit of the present invention, and it is obvious that such changes and modifications fall within the scope of the appended claims.

### Example and Comparative Examples 1 to 6

### Preparation of a Sleeping Pack Comprising a Citron Oil Bead

Table 1 below shows the composition of sleeping pack cosmetic compositions comprising natural citron oil beads.

According to the composition as described in Table 1 below, the citron oil beads were prepared by heating natural citron oils (Citus junos peel oil; from citrons harvested in Goheung, Korea; manufactured by SEOUL PERFUMERY CO., LTD.), behenyl alcohols, jojoba esters, and hydrogenated C₆₋₁₄ polyolefins at 85° C. for 6 minutes, mixing them, and then drying the mixture.

The cosmetic compositions were prepared by mixing the citron oil beads and the ingredients listed in Table 1 below by emulsification, which is a conventional emulsion preparation method.

TABLE 1

| Ingredients | | Comparative | Comparative | Comparative | Comparative | Comparative | Comparative |
|---|---|---|---|---|---|---|---|
| (Unit: weight %) | Example | Example 1 | Example 2 | Example 3 | Example 4 | Example 5 | Example 6 |
| 1.Purified water | Up to | Up to | Up to | Up to | Up to | Up to | Up to |
| | 100 | 100 | 100 | 100 | 100 | 100 | 100 |
| 2. Cyclopentasiloxane | 6.30 | 6.30 | 6.30 | 6.30 | 6.30 | 6.30 | 6.30 |
| 3. Butylene glycol | 6.00 | 6.00 | 6.00 | 6.00 | 6.00 | 6.00 | 6.00 |
| 4. Glycerine | 2.10 | 2.10 | 2.10 | 2.10 | 2.10 | 2.10 | 2.10 |
| 5. Trehalose | 2.00 | 2.00 | 2.00 | 2.00 | 2.00 | 2.00 | 2.00 |
| 6. Dimethicone/vinyl | 0.10 | 0.10 | 0.10 | 0.10 | 0.10 | 0.10 | 0.10 |
| dimethicone | | | | | | | |
| crosspolymer | | | | | | | |
| 7. Dimethiconol | 0.20 | 0.20 | 0.20 | 0.20 | 0.20 | 0.20 | 0.20 |
| 8. Disodium EDTA | 0.02 | 0.02 | 0.02 | 0.02 | 0.02 | 0.02 | 0.02 |
| 9. Ammonium | 0.50 | 0.50 | 0.50 | 0.50 | 0.50 | 0.50 | 0.50 |
| acryloyldimethyltaurate/VP | | | | | | | |
| copolymer | | | | | | | |
| 10. Carbomer | 0.20 | 0.20 | 0.20 | 0.20 | 0.20 | 0.20 | 0.20 |
| 11. Tromethamine | 0.40 | 0.40 | 0.40 | 0.40 | 0.40 | 0.40 | 0.40 |
| 12. Polysorbate 20 | 0.60 | 0.60 | 0.60 | 0.60 | 0.60 | 0.60 | 0.60 |
| 13. Natural citron oil | 0.50 | 0.10 | 1.00 | 1.50 | 0.50 | — | — |
| 14. Behenyl alcohol | 0.50 | 0.20 | 1.00 | 1.50 | — | — | — |
| 15. Jojoba ester | 0.50 | 0.10 | 1.50 | 1.50 | — | — | — |
| 16. Hydrogenated | 2.50 | 0.50 | 5.00 | 7.50 | — | — | — |
| C₆₋₁₄ polyolefine | | | | | | | |
| 17. Lavender oil | — | — | — | — | — | — | 0.50 |

### Experimental Example 1

### Confirmation of Formation of a Citron Oil Bead

Whether citron oil beads were formed in example 1 and comparative examples 1 to 3 was visually observed.

As a result, it was confirmed that the optimum citron oil bead, i.e., citron oil bead having a constant particle diameter without cracking was produced in the case of the example in which the weight ratio of the four ingredients of a citron oil, a behenyl alcohol, a jojoba ester, and a hydrogenated C₆₋₁₄ polyolefin was 1:1:1:5. At this time, it was confirmed that the produced citron oil bead had a particle diameter ranging from 0.05 to 0.8 cm.

In comparative example 1 in which the weight ratio of the four ingredients included in the citron oil beads was 1:2:1:5 and the content of the four ingredients included in the cosmetic composition was 0.9% by weight, it was confirmed that since the weight ratio of the behenyl alcohol among the four ingredients was high and the sum of the four ingredients was small, compared with example 1, the formation of citron oil beads was poor. At this time, it was confirmed that the produced citron oil bead had a particle diameter ranging from 0 to 0.05 cm, and the particle diameter of 0 cm means that the bead itself was not formed. Some formed beads have small particle diameter, and thus may not be good for the preference for the fragrance.

Further, in comparative example 2 in which the weight ratio of the four ingredients included in the citron oil beads was 1:1:1.5:5 and the content of the four ingredients included in the cosmetic composition was 8.5% by weight, it was confirmed that since the weight ratio of the jojoba ester among the four ingredients was high, compared with example 1, the formation of citron oil beads was poor. At this time, some formed citron oil beads have a particle diameter ranging from 0.4 cm to 1 cm, and the beads having a slightly larger particle size is also formed, so that the feeling of use of the cosmetic composition may not be good.

Furthermore, in comparative example 3 in which the weight ratio of the four ingredients included in the citron oil beads was 1:1:1:5 as in example 1, it was confirmed that since the content of the four ingredients included in the cosmetic composition was high as 12% by weight, the formation of citron oil beads was poor. At this time, some formed citron oil beads have a particle diameter ranging from 0.5 cm to 1.2 cm, and the beads having a slightly larger particle size is also formed, so that the feeling of use of the cosmetic composition may not be good.

### Experimental Example 2

### Study of the Preference for the Fragrance and the Effect of Stabilizing the Mind and Body According to the Form (Beads or Oils) of Natural Citron Oils

In order to confirm the preference for the fragrance, fragrance strength, and the effect of stabilizing the mind and body according to the form of natural citron oils, i.e., natural citron oil beads or natural citron oils, an experiment was conducted for the example of a sleeping pack comprising a natural citron oil bead, and comparative example 4 of a sleeping pack comprising a non-beaded natural citron oil.

Evaluation methods, evaluation criteria, and evaluation results are as follows:

### 1. Evaluation Methods

### (1) Subject

The study subjects were 35 healthy, 20 to 30-year-old women who did not take any medication and did not have anosimia. The subjects were instructed to refrain from excessive exercise or excessive drinking on the day before the experiment and to refrain from smoking, beverages, drugs, and gum, which could affect the central nervous system and olfactory sense on the day of the experiment.

### (2) Experimental Samples

As experimental samples, a sleeping pack comprising a natural citron oil bead according to the present invention (example) and a sleeping pack comprising a non-beaded natural citron oil (comparative example 4) were used. In the experiment, the method of directly applying the pack to the face after washing the face and absorbing the natural citron oil was used in the same way, and the questionnaire was made after the natural citron oil was absorbed.

### (3) Experimental Conditions

The experiment was carried out at the evaluation booth of the fragrance laboratory 3 of the AMOREPACIFIC customer research center, and in order to maintain a comfortable evaluation environment during the experiment, the fresh external air was continuously supplied and the temperature and humidity of the evaluation room were maintained at 24° C. and 40%, respectively.

### (4) Experimental Method

The sleeping packs of the example and comparative example 4 were used in random order, and after completely removing the existing fragrance by washing the face with a fragrance-free cleansing foam prior to use products, the sleeping packs were applied to the entire face and then the questionnaire was made. Taking into account fatigue, after 20 minutes break from the end of the evaluation of one product, the next product was evaluated in the same way.

### (5) Analysis of Results

The scores of the questionnaire prepared by the subjects were input, and a paired t-test of the example and comparative example 2 was conducted using the JMP program to analyze the questionnaire results and verify the significance.

### 2. Evaluation Criteria

### (1) Questionnaire for Evaluation of the Preference for the Fragrance

Question: Please evaluate the preference for the fragrance after applying the sleeping pack to the entire face. Please indicate the score corresponding to the following evaluation criteria for the corresponding preference for the fragrance.
Evaluation Criteria
1 point: Very disliked
2 points: Quite disliked
3 points: Slightly disliked
4 points: Normal
5 points: A little liked
6 points: Quite liked
7 points: Very liked

### (2) Evaluation of the Residual Fragrance Strength

Question: Please evaluate the residual fragrance strength 10 minutes after applying the sleeping pack. Please indicate the score corresponding to the following evaluation criteria for how strongly the fragrance feels.
Evaluation Criteria

1 point: Very weak

2 points: Pretty weak

3 points: Slightly weak

4 points: Normal

5 points: A little strong

6 points: Pretty strong

7 points: Very strong

### (3) Evaluation of the Image for the Overall Fragrance

Question: This item evaluates the image of the overall fragrance felt after using the sleeping pack. Please indicate the score corresponding to the following evaluation criteria for the evaluation items (items \textcircled{1} to \textcircled{8}) provided below.
Evaluation Items
\textcircled{1} Feeling of relieving fatigue
\textcircled{2} Feeling receiving comfort
\textcircled{3} Feeling filled with energy
\textcircled{4} Feeling of worry disappearing
\textcircled{5} Feeling of being able to fall asleep comfortably
\textcircled{6} Feeling happy
\textcircled{7} Feeling of being relieved of stress
\textcircled{8} Feeling of being psychologically stable
Evaluation Criteria
1 point: Not feeling that way at all
2 points: Not feeling that way
3 points: Not feeling a little like that
4 points: Usually feeling that way
5 points: Feeling a little bit like that
6 points: Feeling that way
7 points: Feeling very like that

### 3. Evaluation Results

(1) Preference for the Fragrance

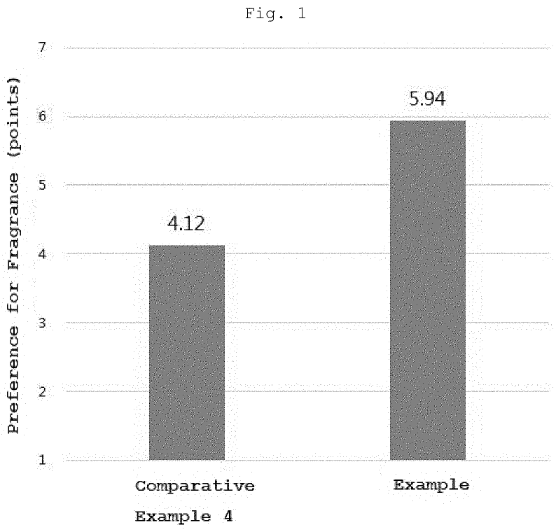

FIG. 1 is a graph showing the evaluated results of the preference for the fragrance for the sleeping packs prepared in the example and comparative example 4.

Referring to FIG. 1, it was confirmed that the preference for the fragrance for the example comprising a natural citron oil bead is much higher than that of comparative example 4 comprising a non-beaded natural citron oil. In addition, it was confirmed that all of the 35 subjects who participated in the evaluation showed high favorability for the example of the sleeping pack comprising a natural citron oil bead.

### (2) Evaluation of the Residual Fragrance Strength

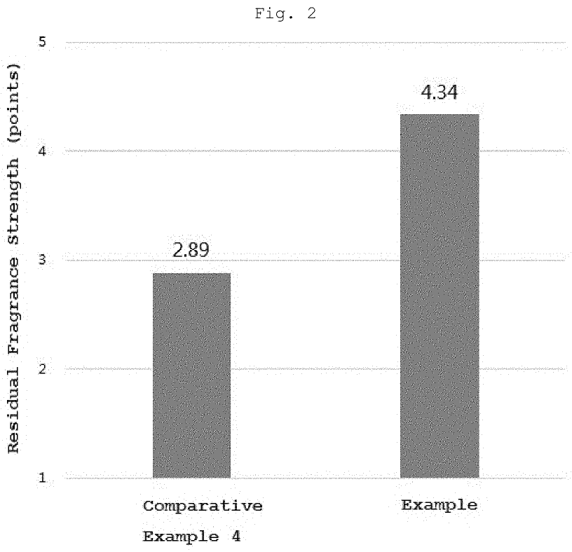

FIG. 2 is a graph showing the evaluated results of the residual fragrance strength for the sleeping packs prepared in the example and comparative example 4.

Referring to FIG. 2, it was confirmed that even in the evaluation of the residual fragrance strength 10 minutes after applying the surface pack to the entire face, the fragrance of the citron was felt more strongly in the sleeping pack comprising the natural citron oil bead of the example.

### (3) Evaluation of the Images for the Overall Fragrance

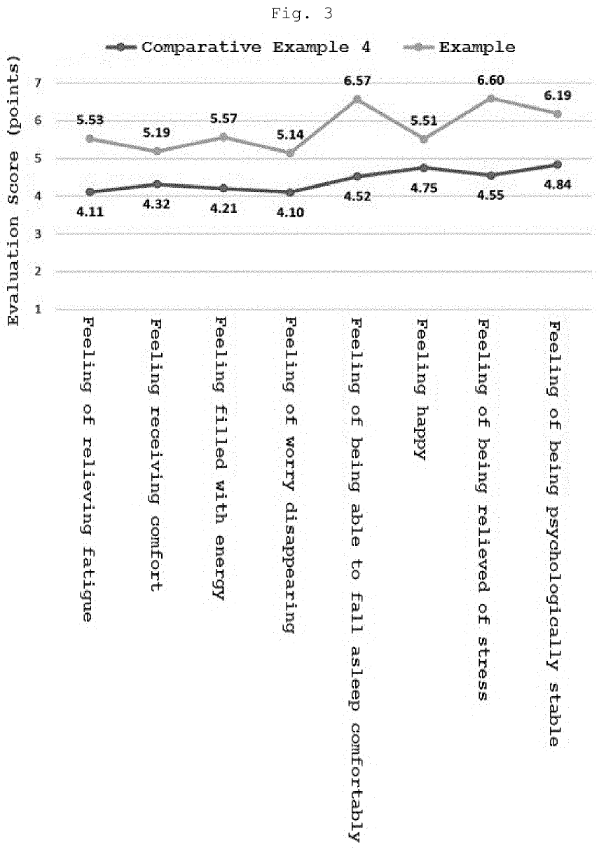

FIG. 3 is a graph showing the evaluated results of the image for the overall fragrance for the sleeping packs prepared in the example and comparative example 4.

Referring to FIG. 3, it was confirmed that the sleep pack comprising the natural citron oil bead of the example showed a significantly higher score than the sleep pack comprising the non-beaded natural citron oil of comparative example 4, in the items such as “feeling of being relieved of stress,” “feeling of being psychologically stable,” and “feeling of being able to fall asleep comfortably,” and thus the sleep pack comprising the natural citron oil bead of the example induced a comfortable sleeping, together with the effects of relieving the stress and stabilizing the mind and body.

Experimental Example 3

### Evaluation of the Deep Sleep Effect of the Sleep Packs Depending on the Presence and Absence of the Fragrance and the Kind of the Fragrance

In order to confirm the deep sleep effect of sleep packs depending on the presence and absence of the fragrance and the kind of the fragrance, experiments were conducted for the sleeping packs of the example and comparative examples 5 and 6.

The example is for a sleeping pack comprising a natural citron oil bead and having a citron fragrance, comparative example 5 is for a fragrance-free sleeping pack that does not comprise the natural oil bead, and comparative example 6 is for a sleeping pack comprising a lavender oil and having a lavender fragrance, which is generally known to have the deep sleep effect.

In addition, the deep sleep effect for the case of not using a sleeping pack was also evaluated as a control group.

Evaluation methods, evaluation criteria, and evaluation results are as follows:

### 1. Evaluation Methods

(1) Subject

The study subjects were 22 healthy, 20 to 30-year-old women who did not take any medication and did not have anosimia. The subjects were instructed to refrain from excessive exercise or excessive drinking on the day before the experiment and to refrain from smoking, beverages, drugs, and gum, which could affect the central nervous system and olfactory sense on the day of the experiment. This experiment was conducted for adult women and it was reported that there was a correlation between the sensory change of the fragrance according to the sexual cycle of the woman, i.e., the female menstrual cycle and mood, and thus the menstrual cycles of the subjects were considered.

### (2) Experimental Samples

As the experimental samples, a sleeping pack comprising the natural citron oil bead (example) as the present invention, and a fragrance-free sleeping pack (comparative example 5) and a sleeping pack comprising a lavender oil, which is well known to have the deep sleep effect (comparative example 6) as the comparative examples were used, and the control group was defined as when no pack was used.

The experiment was carried out under four conditions of without using packs and using three kinds of sleeping pack samples. The three conditions except the condition of without using the pack were the same as the method of directly applying the pack to the face after washing the face and absorbing the oil, and the sleep was taken after the oil was absorbed. A polysomnography was performed by measuring electroencephalogram (EEG), electrooculogram (EOG), chin electromyography (EMG), and electrocardiography (ECG) during sleep.

### (3) Experimental Conditions

This experiment was carried out in the sleep laboratory (4.8 m×3 m×2.4 m) constructed by the Korea Research Institute of Standards and Science, and a soundproofing device was installed inside the sleep laboratory to exclude the influence of the external environment during the experiment. The sleep laboratory was kept at constant temperature and humidity, and the temperature was 24° C. and the humidity was 40%.

### (4) Measurement Method

For physiological signals during sleep, EEG, EOG, chin EMG, and ECG were measured using a polysomnography device and the body movement was measured using Actiheart of Mini Mitter Company, Inc.

### (5) Experiment Procedure

The experiment was carried out under four conditions of without using packs and using three kinds of pack samples and was the same as the method of directly applying the pack to the face after washing the face and absorbing the oil, and the sleep was taken after the oil was absorbed. The type of sample was randomized for each subject in order to exclude the order effect on the sleep, and sleep was taken 1-2 times in the sleep laboratory for adaptation to the sleep laboratory prior to this experiment. Sleep time was taken for 2 hours during the day, and each subject was asked to sleep at the same time.

### (6) Analytical Method

While the subjects were taking their sleep, physiological signals were measured using a polysomnography device and sleep stages were analyzed using the measured signals. The determination of the sleep stages was performed according to the method of Rechtschaffen and Kales (1968), which is an international standard.

Using the divided sleep stages, the sleep latency and sleep efficiency were compared, and the number of body movements for each condition was also compared. Statistical analysis was performed with ANOVA and t-test using the SPSS program, and a post-hoc test was made using the Tukey's method.

### 2. Evaluation Criteria and Evaluation Results

(1) Sleep Latency

Sleep latency is the time from going into bed to sleeping, and the difference in time (minutes) to reach sleep depending on the samples was evaluated.

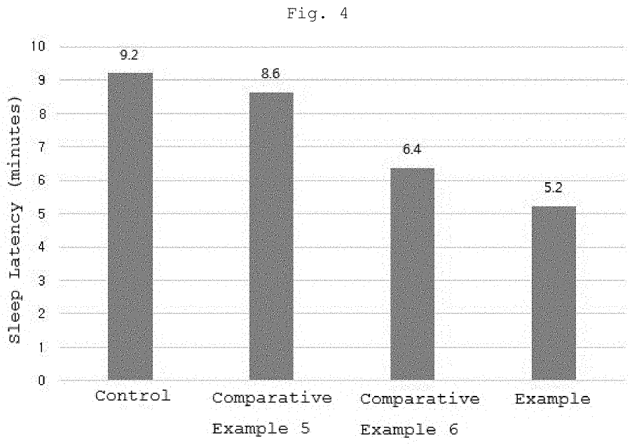

FIG. 4 is a graph showing the evaluated results of sleep latency for the sleeping packs prepared in the example and comparative examples 5 and 6.

Referring to FIG. 4, it was confirmed that the average sleep latency was the longest to reach sleep in the control condition without any sleeping pack and it was asleep in the earliest time in the example comprising the natural citron oil bead.

### (2) Sleep Efficiency

Sleep efficiency is the ratio of the total sleep time to the time on the bed, and the ratio of sleep time taken during the experiment was compared.

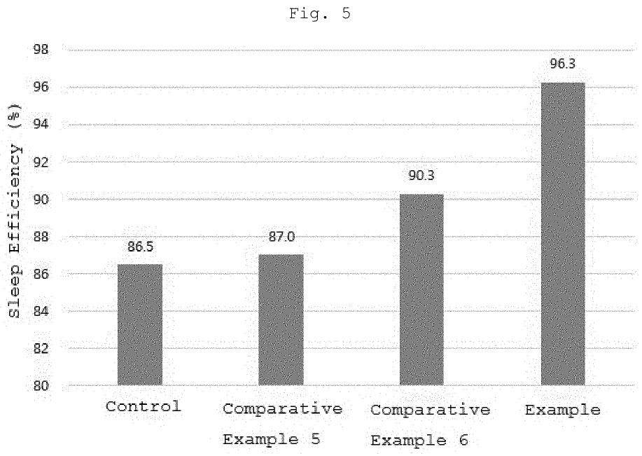

FIG. 5 is a graph showing the evaluated results of sleep efficiency for the sleeping packs prepared in the example and comparative examples 5 and 6.

Referring to FIG. 5, it was confirmed that the average sleep efficiency was the lowest in the control group and it was in the sleep for the longest time in the example comprising the natural citron oil bead.

### (3) Body Movement

The body movement is the number of times the subject moves during sleep, and the number of times the subject moved during the sleeping time was measured using a device for measuring body movements (pedometer, etc.).

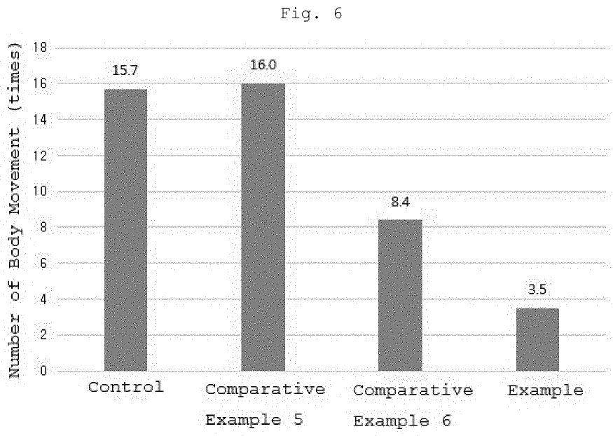

FIG. 6 is a graph showing the measured results of the number of body movement during sleeping when the sleeping packs prepared in the example and comparative examples 5 and 6 are used.

Referring to FIG. 6, the condition of comparative example 5 showed body movement of about 16 times during the experimental time and exhibited the greatest number of body movement among the four conditions, but the condition of the examples comprising the natural citron oil bead of the present invention exhibited significantly less body movement than other conditions during sleep.

1. A cosmetic composition comprising a citron oil bead.

2. The cosmetic composition according to claim 1, wherein the citron oil bead comprises a citron oil, a behenyl alcohol, a jojoba ester, and a hydrogenated C₆₋₁₄ polyolefin.

3. The cosmetic composition according to claim 2, wherein the citron oil bead comprises 10 to 140 parts by weight of behenyl alcohol, 10 to 140 parts by weight of jojoba ester, and 300 to 1000 parts by weight of hydrogenated C₆₋₁₄ polyolefin, based on 100 parts by weight of the citron oil.

4. The cosmetic composition according to claim 1, wherein the citron oil bead is comprised in an amount of 2.0 to 10.0% by weight, based on the total weight of the cosmetic composition.

5. The cosmetic composition according to claim 1, wherein the particle diameter of the citron oil bead is 0.05 to 0.8 cm.

6. The cosmetic composition according to claim 1, wherein the cosmetic composition is formulated into a skin softener, a nutritional toner, an emulsion, a massage cream, a nutritional cream, a pack, a gel, skin adhesive type cosmetics, a lotion, an ointment, a gel, a cream, a patch, or a spray formulation.

7. A method for inducing deep sleep, comprising applying a cosmetic composition comprising a citron oil bead.

8. The method of claim 7, wherein the cosmetic composition is applied onto the skin once a day for 3 to 8 weeks.

9. The method of claim 7, wherein the cosmetic composition is applied onto the skin before sleeping.
